(19) United States
(12) Patent Application Publication
(10) Pub. No.: US 2005/0196857 A1
(43) Pub. Date: Sep. 8, 2005

(54) Live cell chamber for microscopes

(76) Inventor: Yongjin Lee, Seoul (KR)

Correspondence Address: PARK & SUTTON LLP, 3255 WILSHIRE BLVD, SUITE 1110, LOS ANGELES, CA, 90010, US

(21) Appl. No.: 11/069,009

(22) Filed: Mar. 1, 2005

(30) Foreign Application Priority Data
Mar. 6, 2004 (KR) 10-2004-0015271

(51) Int. Cl. C12M 1/22

(52) U.S. Cl. 435/292.1; 435/293.1; 435/305.4; 359/398

(57) ABSTRACT

Disclosed herein is a live cell chamber for a microscope. The live cell chamber comprises a chamber body, a transparent specimen plate, a specimen mounting base, and at least one sealing member. The chamber body includes at least one vertically perforated inner space, and a plurality of magnets. The specimen mounting base includes a specimen receiving portion, a magnetic member embedded around the specimen receiving portion such that the magnetic member can react to the magnets, and at least one through hole formed through the specimen receiving portion such that the center of the through hole is collinear with the center of the inner space of the chamber body. The sealing member is attached to the one end of the inner space while being attached at the other side to an upper surface of the specimen plate, thereby sealing a space defined between the inner space and the plate.

### CLAIMING FOREIGN PRIORITY

The applicant claims and requests a foreign priority, through the Paris Convention for the Protection of Industry Property, based on a patent application filed in the Republic of Korea (South Korea) with the filing date of Mar. 6, 2004, with the patent application number 10-2004-0015271, by the applicant. (See the Attached Declaration).

### BACKGROUND OF THE INVENTION

1. Field of the Invention

The present invention relates to a specimen mount for microscopes, and, more particularly, to a live cell chamber for microscopes.

2. Description of the Related Art

A microscope generally comprises an ocular lens, a body tube, a revolving nosepiece, an objective lens, a macroscrew, a microscrew, a stand, a stage, a reflector, and the like.

In order to observe a certain specimen using a microscope, the specimen must be located on the stage of the microscope. In this way, for a specimen of dead cells, the specimen is attached to a glass slide, and is then observed on the stage in a state of being covered by a cover glass (or specimen glass) placed thereupon. On the other hand, for a specimen of live cells, it is necessary to provide a live cell chamber that enables the specimen to be alive for a predetermined period. Conventionally, the live cell chamber has employed a hook or screw type construction in order to couple the cover glass (specimen glass) to the chamber.

For a hook type live cell chamber, the live cell chamber comprises a chamber body formed at the center with a stepped depression and a perforated inner space while being formed at the top surface with a plurality of hooks, a plurality of glass plates and O-rings inserted into the depression of the chamber body, and a body coupler formed at an outer peripheral surface with a plurality of grooves coupled to the hooks of the chamber body while being formed at the center with a through hole.

For a screw type live cell chamber, the live cell chamber comprises a chamber body formed at the center with a stepped depression and a perforated inner space while being formed on a sidewall of the depression with screw grooves, one or more glass plates and O-rings inserted into the chamber body, and a body coupler formed on an outer peripheral surface with threads coupled to the screw grooves of the chamber body while being formed at the center with an through hole.

As such, the conventional hook-type or screw type live cell chamber is adapted to allow the glass plate to be coupled to the chamber body through the hook or screw structure, so that over pressure or vibration arising upon coupling of the hooks or the screw result in frequent damage to the glass plate and incomplete coupling between the glass plate and the chamber body, thereby causing frequent leakage of liquid within the chamber.

Furthermore, since the live cell chamber as described above requires a number of components for coupling, and has a complicated construction, these chambers are not only difficult to operate, but also require extensive times for mounting the specimen, thereby critically influencing survival of the specimen and experimental environment. Additionally, since the specimen is observed with an upper portion of the chamber being opened, it is necessary to input excess amount of mixture of gases into the chamber in order to satisfy the experimental requirements for survival of the specimen, thereby significantly increasing operating costs of the live cell chamber.

### SUMMARY OF THE INVENTION

The present invention has been made to solve the above problems, and it is an object of the present invention to provide a live cell chamber for microscopes, designed to have a reduced number of components, allow some components to be coupled to each other through magnetic force, and to provide a chamber body having a closable upper portion in order to allow a survival environment of a specimen to be created only within the chamber, thereby simplifying the construction of the live cell chamber, allowing easy and rapid mounting of a specimen therein, and reducing operating costs of the live cell chamber.

In accordance with an aspect of the present invention, the above and other objects can be accomplished by the provision of a live cell chamber for a microscope, comprising: a chamber body including at least one vertically perforated inner space, and a plurality of magnets embedded around the inner space in the chamber body; a transparent specimen plate for locating the specimen thereon; a specimen mounting base including a specimen receiving portion for seating the specimen plate, a magnetic member embedded around the specimen receiving portion such that the magnetic member can react to the magnets of the chamber body so as to allow the specimen mounting base to be attached to the chamber body, and at least one through hole formed through the specimen receiving portion such that the center of the through hole is collinear with the center of the inner space of the chamber body when the specimen mounting base is coupled at one end of the inner space to the chamber body; and at least one sealing member attached at one side to an outer peripheral surface of the one end of the inner space of the chamber body while being attached at the other side to an upper surface of the specimen plate seated on the specimen mounting base, thereby sealing a space defined between the one end of the inner space and the upper surface of the plate.

### BRIEF DESCRIPTION OF THE DRAWINGS

The foregoing and other objects and features of the present invention will be more clearly understood from the following detailed description taken in conjunction with the accompanying drawings, in which:

FIG. 1a is an exploded front perspective view of a live cell chamber according to the present invention;

FIG. 1b is an exploded rear perspective view of the live cell chamber according to the present invention;

FIG. 1c is a transverse cross section of a heater line formed to a chamber body;

FIG. 2a is a front perspective view of the live cell chamber in an assembled state; and

FIG. 2b is a rear perspective view of the live cell chamber in the assembled state.

### DESCRIPTION OF THE PREFERRED EMBODIMENTS

Preferred embodiments will now be described in detail with reference to the accompanying drawings.

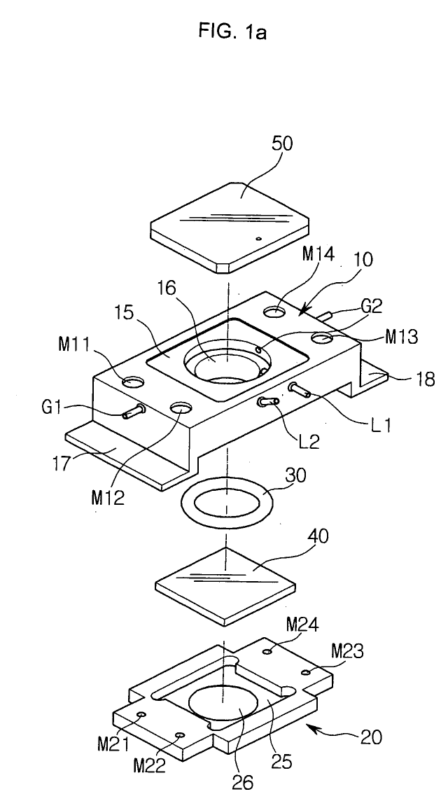

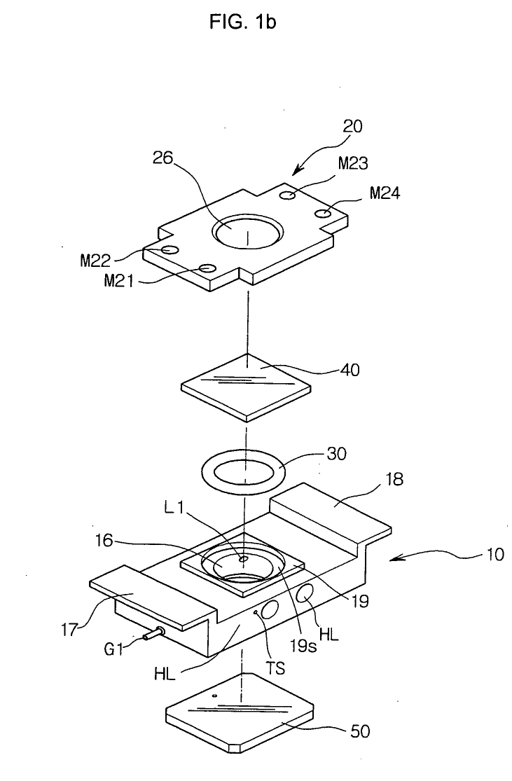

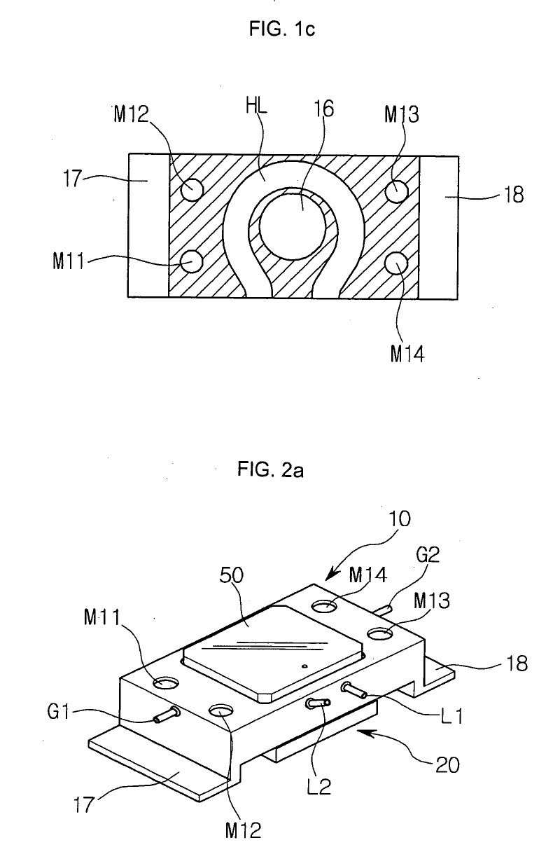

FIG. 1a is an exploded front perspective view of a live cell chamber according to the present invention. Referring to FIG. 1a, the live cell chamber comprises a chamber body 10, a specimen mounting base 20, an O-ring 30, a specimen glass 40, a transparent cover 50, and the like.

The chamber body 10 has a parallelepiped shape, and a vertically perforated cylindrical inner space 16 defined therein. A cover mounting portion 15 having a stepped planar surface is formed at one end of the inner space 16 such that the transparent cover 50 can be seated thereon. The height of the cover mounting portion 15 may be the same as the thickness of the transparent cover 50, so that, when the transparent cover 50 is seated on the cover mounting portion 15, the top surface of the transparent cover 50 may be coplanar with the top surface of the chamber body 10.

The chamber body 10 has body seating legs 17 and 18 for seating the chamber body 10 onto the stage, each of which is formed at either lower end thereof and is extended downwardly and laterally in a plate shape, thereby forming an “L”-shape. The body seating legs 17 and 18 may have a height adapted such that, when the specimen mounting base 20 is coupled to a lower side of the chamber body 10, the bottom surface of the chamber body 10 is coplanar with the specimen mounting base 20.

A plurality of magnets M11, M12, M13 and M14 are embedded around the inner space 16 in the chamber body 10. Preferably, the magnets M11, M12, M13 and M14 are embedded in the chamber body 10 so as to be adjacent to a portion where the specimen mounting base 20 is coupled to the chamber body 10, that is, to the lower side of the chamber body 10.

The chamber body 10 is provided with a gas supply pipe G1 for supplying gas, such as CO₂, into the inner space 16, and a gas discharge pipe G2 for discharging the gas from the inner space 16 to the outside, which are provided into the inner space 16 through one side of the chamber body 10. Preferably, the gas supply and discharge pipes G1 and G2 are equipped at opposite sides of the chamber body 10, respectively.

The chamber body 10 is provided with a liquid supply pipe L1 for supplying a liquid for specimen observation into the inner space 16, and a liquid discharge pipe L2 for discharging the liquid from the inner space 16 to the outside, which are provided into the inner space 16 through one side of the chamber body 10. Preferably, the liquid supply pipe L1 is formed into an upper portion of the inner space 16 through the side of the chamber body 10, and a liquid discharge pipe L2 is formed in a lower portion of the inner space 16 through the side of the chamber body 10.

The specimen mounting base 20 is a rectangular plate, and is coupled to the lower side of the chamber body 10 between the body seating legs 17 and 18. A specimen receiving portion 25 having a stepped planar surface is formed on an upper side of the specimen mounting base 20, at which the specimen mounting base 20 is coupled to the chamber body 10, such that the specimen glass 40 can be seated thereon. The specimen mounting base 20 has a through hole 26 formed at the center thereof so as to allow the interior of the chamber to be observed from the outside.

A plurality of magnets M21, M22, M23 and M24 are embedded around the specimen receiving portion 25 in the specimen mounting base 20. The magnets M21, M22, M23 and M24 of the specimen mounting base 20 are located corresponding to the magnets M11, M12, M13 and M14 of the chamber body 10, respectively. Furthermore, the magnets of the specimen mounting base 20 and the magnets of the specimen mounting base 20 are positioned such that the n-magnetic pole of each magnet corresponds to the s-magnetic pole of an associated magnet. Alternatively, as with the magnets M21, M22, M23 and M24 embedded in the specimen mounting base 20, the specimen mounting base 20 may comprise a certain metallic magnetic member which can react to the magnets M11, M12, M13 and M14 of the chamber body 10, so that the specimen mounting base 20 can be attached to the chamber body 10 by virtue of magnetic force between the magnets embedded in the chamber body 10 and the magnetic member embedded in the specimen mounting base 20 when being located to the lower side of the chamber body 10.

The O-ring 30 is brought into close contact with a lower outer peripheral portion of the inner space 16 of the chamber body 10 and to an upper surface of the specimen glass 40 when the chamber body 10 is coupled with the specimen mounting base 20, thereby constituting a sealed space defined by the inner space 16 of the chamber body 10 and the specimen glass 40. The O-ring 30 may be made of an elastic material, and preferably made of a silicon material in view of the characteristics of observation of a live specimen.

The specimen glass 40 is a glass member on which the specimen is seated at the center thereof, and is fitted onto the specimen receiving portion 25 of the specimen mounting base 20. The specimen glass 40 is made of a transparent material, such as glass.

The transparent cover 50 is seated on the cover receiving portion 15 of the chamber body 10, and acts to seal the upper portion of the inner space 16 of the chamber body 10. The transparent cover 50 is made of a transparent material, such as glass, so as to allow the inner space 16 and the specimen to be observed from the outside.

FIG. 1b is an exploded rear perspective view of the live cell chamber according to the invention. Referring to FIG. 1b, the chamber body 10 is formed around the center of the lower surface thereof with a raised feature 19 that is fitted onto the specimen receiving portion 25. The raised feature 19 is shaped such that it can be fixedly fitted onto the specimen receiving portion 25, and has a height the same as the depth of the specimen receiving portion 25. The raised feature 19 is formed at the center thereof with an O-ring step 19s, such that the O-ring 30 is fitted onto the O-ring step 19s. The depth of the O-ring step 19s is smaller than the thickness of the O-ring 30, so that even after the O-ring 30 is fitted onto the O-ring step 19s, the O-ring 30 is raised a predetermined height over the upper surface of the raised feature 19. This construction ensures that the O-ring 30 is brought into intimate contact with the specimen glass 40. The chamber body 10 is provided with a heater line HL, which surrounds the inner space 16 and is in communication with the outside, for maintaining the inner space 16 at a constant temperature, and a sensor hole SH to which a temperature sensor TS for measuring the temperature of the inner space 16 is inserted.

FIG. 1c is a transverse cross section of the heater line formed to the chamber body. Although the heater line HL may have various shapes, such as a “C”-like shape, a parallel-line (∥) shape, and the like, it preferably has a loop shape, as shown in FIG. 1c, which surrounds the inner space 16 in order to effectively transfer heat into the inner space 16.

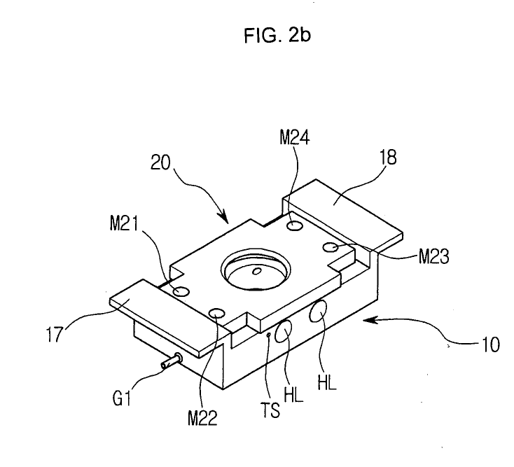

FIG. 2a is a front perspective view of the live cell chamber in an assembled state, and FIG. 2b is a rear perspective view of the live cell chamber in the assembled state.

Referring to FIG. 2a, the transparent cover 50 is fitted onto the cover receiving portion 25 of the chamber body 10, and the specimen mounting base 20 is coupled to the lower side of the chamber body 10. Although it is desirable that the upper surface of the transparent cover 50 is coplanar with the upper surface of the chamber body 10 when the transparent cover 50 is fitted onto the cover receiving portion 25 of the chamber body 10, the upper surface of the transparent cover 50 may be raised above the upper surface of the chamber body 10.

Referring to FIG. 2b, the specimen mounting base 20 is fitted onto the lower side of the chamber body 10 between the body seating legs 17 and 18, and at this time, the bottom surface of the specimen mounting base 20 is coplanar with the bottom surface of the chamber body 10 so as to allow the live cell chamber to be mounted on the stage.

In the above description, although the inner space 16 of the chamber body 10 has been described as having the cylindrical shape and being defined as a single space, it should be noted that the present invention is not limited to this construction including the shape and the number of the components. Thus, the inner space 16 may have various shapes, such as a triangular shape, a rectangular shape, a pentagonal shape, etc. Additionally, the inner space 16, the O-ring 30 and the through holes may be provided as a plurality of components, respectively, so as to allow a plurality of live cell specimens to be positioned in the plurality of inner spaces. Similarly, although the raised feature 19 formed on the lower side of the chamber body 10 may have a circular shape, it preferably has a polygonal shape in order to prevent the specimen mounting base 20 from rotating when being coupled to the chamber body 10. Moreover, since the O-ring 30 made of the elastic material can be resiliently deformed when being fitted onto the O-ring step 19s, the O-ring step 19s of the raised feature 19 may have various shapes including a rectangular shape.

A method of using the live cell chamber having the construction as described above will be described hereinafter. First, after a specimen is located on the specimen glass 40, the specimen glass 40 is fitted to the specimen receiving portion 25 of the specimen mounting base 20. Then, as the specimen mounting base 20 is fitted to the lower side of the chamber body 10, the specimen mounting base 20 is attached to the chamber body 10 by virtue of magnetic force between the magnets M21, M22, M23 and M24 embedded in the chamber body 10 and the magnets M21, M22, M23 and M24 embedded in the specimen mounting base 20. At this time, the specimen mounting base 20 is prevented from rotating or vibrating through engagement between the chamber body 10 and the specimen mounting base 20, that is, between the raised feature 19 of the chamber body 10 and the specimen receiving portion 25 of the specimen mounting base 20.

When the raised feature 19 is engaged with the specimen receiving portion 25, the O-ring 30 is resiliently attached to the specimen glass 40, thereby sealing the lower portion of the inner space 16. Then, the transparent cover 50 is brought into intimate contact with the cover receiving portion 15 of the chamber body 10, so that the upper portion of the inner space 16 is closed.

Gas for survival of the specimen is supplied to or discharged from the inner space 16 through the gas supply pipe G1 or the gas discharge pipe G2, and liquid for specimen observation is supplied to or discharged from the inner space 16 through the liquid supply pipe L1 or the liquid discharge pipe L2.

It should be noted that the above embodiment is illustrated as an example. Accordingly, various modifications can be made to the chamber body and the specimen mounting base, including various shapes, such as a circular shape and the like.

As apparent from the description, the live cell chamber of the invention has the reduced number of components, thereby providing a simple construction, allows the specimen mounting base to be easily coupled to the chamber body, and particularly, has the chamber body, the upper portion of which can be closed so that the environment for the live cell is created only within the chamber body, thereby allowing operating costs of the live cell chamber to be reduced.

It should be understood that the embodiments and the accompanying drawings have been described for illustrative purposes and the present invention is limited by the following claims. Further, those skilled in the art will appreciate that various modifications, additions and substitutions are allowed without departing from the scope and spirit of the invention as set forth in the accompanying claims.

1. A live cell chamber for a microscope, comprising:
a chamber body including at least one vertically perforated inner space, and a plurality of magnets embedded around the inner space in the chamber body;
a transparent specimen plate for locating the specimen thereon;
a specimen mounting base including a specimen receiving portion for seating the specimen plate, a magnetic member embedded around the specimen receiving portion such that the magnetic member can react to the magnets of the chamber body so as to allow the specimen mounting base to be attached to the chamber body, and at least one through hole formed through the specimen receiving portion such that the center of the through hole is collinear with the center of the inner space of the chamber body when the specimen mounting base is coupled at one end of the inner space to the chamber body; and
at least one sealing member attached at one side to an outer peripheral surface of the one end of the inner space of the chamber body while being attached at the other side to an upper surface of the specimen plate seated on the specimen mounting base, thereby sealing a space defined between the one end of the inner space and the upper surface of the plate.

2. The live cell chamber as set forth in claim 1, wherein the live cell chamber further comprises a transparent cover, and the chamber body is formed with a cover receiving portion depressed a predetermined depth around the other end of the inner space such that, when the transparent cover is seated on the cover receiving portion, the transparent cover closes the other end of the inner space of the chamber body.

3. The live cell chamber as set forth in claim 2, wherein the chamber body further includes a gas supply pipe for supplying gas for survival of the specimen into the inner space, a gas discharge pipe for discharging the gas from the inner space to the outside, a liquid supply pipe for supplying a liquid for specimen observation into the inner space, and a liquid discharge pipe for discharging the liquid from the inner space to the outside, the pipes being formed from the outside into the inner space through the chamber body.

4. The live cell chamber as set forth in claim 3, wherein the chamber body is further formed therein with a heater line around the inner space to maintain the inner space at a constant temperature, and at one side of the inner space with a sensor hole to which a temperature sensor for measuring the temperature of the inner space is inserted.

5. The live cell chamber as set forth in claim 4, wherein the chamber body is further formed around the one end thereof with a raised feature fitted to the specimen receiving portion of the specimen mounting base.

6. The live cell chamber as set forth in claim 5, wherein the raised feature is a raised step surface, and the specimen receiving portion is a stepped depression onto which the raised feature is fitted.

7. The live cell chamber as set forth in claim 6, wherein the raised feature and the specimen receiving portion have a polygonal outer periphery.

8. The live cell chamber as set forth in claim 7, wherein the raised feature is formed with a depressed step onto which the sealing member is fitted.

9. The live cell chamber as set forth in claim 1, wherein the sealing member is an elastic silicon O-ring.

10. The live cell chamber as set forth in claim 9, wherein the magnetic material is a magnet.
